(19) United States
(12) Patent Application Publication
(10) Pub. No.: US 2025/0154698 A1
(43) Pub. Date: May 15, 2025

(54) PROCESS FOR PRODUCTION OF CLOTH PRODUCTS IMPREGNATED WITH AN IMPREGNATING AGENT

(71) Applicant: DR. SCHUMACHER GMBH, Malsfeld (DE)

(72) Inventors: Wojciech Siwek, Luba´n (PL); Anke Birkelbach, Malsfeld (DE); Mina Heidari, Malsfeld (DE)

(73) Assignee: DR. SCHUMACHER GMBH, Malsfeld (DE)

(21) Appl. No.: 18/939,773

(22) Filed: Nov. 7, 2024

(30) Foreign Application Priority Data
Nov. 9, 2023 (DE) 102023131186.7

(51) Int. Cl. D04H 1/492; D04H 1/4374

(52) U.S. Cl. CPC D04H 1/492; D04H 1/4374; D10B 2509/00

(57) ABSTRACT

The invention relates to a process for the production of cloth products impregnated with an impregnating agent, wherein the process comprises reinforcing and/or bonding fibres and/or nonwovens by means of hydroentanglement, wherein after the hydroentanglement a concentrate of an impregnating agent is applied to the fibres and/or nonwovens which are interconnected and/or reinforced and are still impregnated with water, wherein the concentrate, together with water with which the interconnected and/or reinforced fibres and/or nonwovens are impregnated after hydroentanglement, forms the impregnating agent with which the impregnated cloth products are impregnated.

The present invention relates to a new process for production of cloth products that are impregnated with an impregnating agent involving reinforcing and/or bonding fibres or nonwovens in the manufacture of cloth products.

Depending on their nature, cloth products are used in a wide variety of applications, in particular in the cosmetic and hygienic field and also for numerous cleaning applications. Depending on the desired application, which is often wet or moist, the cloth products can already be impregnated with an impregnating agent and packaged individually or in units with a predetermined number of cloth products, or they can also be packaged in an unimpregnated state and may only be moistened or impregnated after removal from the packaging, if this is desired or necessary.

Depending on the desired application of the cloth products, these may comprise several layers of nonwoven fabric, which may have the same or different composition(s). In the case of disposable sweeping cloths for cleaning surfaces, it is known that these should comprise several layers of nonwoven fabric, which are usually composed differently. On the one hand, this ensures sufficient stability of the sweeping cloth and, on the other hand, in the case of layers of differently composed nonwovens, can ensure that, for example, a first layer provides good liquid absorption, while a second layer provides good liquid release, if the nonwovens are selected accordingly.

Various processes for producing nonwovens from loose fibres and also for producing multi-layered cloth products by bonding several layers of nonwoven fabric together have long been known from the prior art. Known processes by which a pre-bonded fibre composite can be obtained from loose fibres are, for example, the drylaid process, the wetlaid process, the meltblown process and the spunlaid process. In addition to the aforementioned processes belonging to the ‘web formation’ technology, processes belonging to the ‘web bonding’ technology such as calendering, hydroentanglement, spunlace, needle punch and air through bonding are also used.

In hydroentanglement, loose fibres are entangled with jets of water emitted from nozzles under high pressure, whereby a reinforcement in the form of a fibre composite is obtained. It is also known from PCT/EP2023/072187 that hydroentanglement is also suitable for bonding layers that have already been pre-reinforced, since the high pressure with which the water jets hit the non-woven layers during hydroentanglement means that even fibres from different layers that have already been reinforced to form a fibre composite still form a bond with each other.

In addition to hydroentanglement, other processes for bonding pre-reinforced layers include ultrasonic reinforcement and needling.

DE 10 2021 122 041 B1 describes a multi-layer disposable floor wipe having an abrasive strip, which comprises at least one cleaning layer and one core layer, both of which contain cellulose and consist entirely of natural fibres and/or regenerated fibres, wherein a scratch strip having increased abrasive properties compared to the remaining cleaning layer is arranged on or in the cleaning layer, and wherein the scratch strip contains hemp and also consists entirely of natural fibres and/or regenerated fibres. The cleaning layer and the core layer as well as optional further layers are preferably bonded together by means of micro-entangling by means of movement generated by ultrasound.

DE 101 64 640 A1 describes a process for finishing fluidised-jet reinforced fabrics, in which the textile fabrics are finished with a finishing agent between the fluidised-jet reinforcement and a drying process, for example to achieve desired surface effects or to improve the strength of the textile fabrics.

AT 503 625 A1 describes a combination of a melt-blowing process with a hydroentanglement step without prior drying of the product.

DD 246 129 A1 describes a transport element for the production of nonwovens, the bonding of which is carried out without a binding agent by means of fluid jets under high pressure.

### OBJECT OF THE INVENTION

It is the object of the present invention to provide an alternative process for the production of impregnated cloth products by reinforcing and/or bonding fibres and/or nonwovens, which is particularly suitable for the production of cloth products impregnated with surfactant-containing impregnating agent. This alternative manufacturing process should be characterized in particular by a more energy-and resource-saving process control compared to processes known from the prior art. Preferably, the process should also produce fewer waste products.

### GENERAL DESCRIPTION OF THE INVENTION

The invention achieves this object with the features of the claims and, in particular, with a process for producing cloth products impregnated with impregnating agent, which involves reinforcing and/or bonding fibres and/or nonwovens by means of hydroentanglement, and in which, after hydroentanglement, a concentrate of an impregnating agent is applied to the fibres and/or nonwovens which have been bonded and/or reinforced and are still impregnated with water, wherein the concentrate, together with water with which the bonded and/or reinforced fibres and/or nonwovens are impregnated after hydroentanglement, forms the impregnating agent with which the impregnated cloth products are impregnated.

The fibres used in the process according to the invention can be any fibres of the same or different types, in particular natural fibres as well as synthetic fibres.

The term ‘nonwovens’ is to be understood as reinforced fabrics consisting of fibres in which the fibres have already formed a firm bond. The fibres contained in the nonwovens can also be fibres of the same or different types, in particular natural fibres and synthetic fibres. In embodiments in which two or more nonwovens are to be bonded together by the process according to the invention, the nonwovens are generally composed differently in order to fulfil different functions in the resulting cloth product, whereby each of the nonwovens may contain fibres of the same type or different fibres of any type, which may be natural fibres and/or synthetic fibres independently of one another. This already shows a first advantage of the process according to the invention, because due to the bonding by means of water jets, the composition of the nonwovens is not limited to synthetic fibres, as it would be the case if the bonding of the nonwovens were carried out by thermal means.

In the context of the present invention, ‘bonding’ of fibres or nonwovens by hydroentanglement primarily means entangling or hooking the loose fibres or the fibre ends in a nonwoven fabric into one another, and not a substance-to-substance bond.

In embodiments of the process according to the invention, in which two or more nonwovens are bonded together, the nonwovens used may optionally have been produced by manufacturing technologies belonging to ‘web formation’ technology or ‘web bonding’ technology. Preferred processes belonging to the ‘web formation’ technology are carding, airlaid process, wetlaid process, meltblown process and spunlaid process. Preferred processes belonging to the ‘web bonding’ technology are calendering, hydroentanglement, needle punching and air through bonding.

In the context of the present invention, the idea of reinforcing or bonding loose fibres or already bonded nonwovens to each other by hydroentanglement has been further developed.

In the already known bonding of nonwovens by means of hydroentanglement, in the prior art a drying step is carried out after the hydroentanglement step in order to remove the water with which the fibres are impregnated after hydroentanglement. To obtain cloth products impregnated with impregnating agent, the fibres are then impregnated with the desired impregnating agent in a subsequent third step.

In accordance with the invention, however, the drying step, which in the prior art follows the hydroentanglement, is completely or partially dispensed with, i.e. all or part of the water with which the interconnected and/or reinforced fibres and/or nonwovens are impregnated as a result of hydroentanglement remains there as part of the impregnating agent and, together with the concentrate of an impregnating agent which is applied after hydroentanglement to the interconnected and/or reinforced fibres and/or nonwovens still impregnated with water, forms the impregnating agent with which the impregnated cloth products are impregnated.

In the context of the present invention, a complete or partial omission of the drying step means that, after hydroentanglement, either no drying is carried out at all (complete omission of the drying step) or that the water contained in the interconnected and/or reinforced fibres and/or nonwovens as a result of the hydroentanglement is only partially removed by drying, for example up to 10%, up to 20%, up to 30%, up to 40% or up to 50% (partial/partial omission of the drying step).

By completely or partially dispensing with the drying step following hydroentanglement known from the prior art and applying a concentrate to the bonded and/or reinforced fibres and/or nonwovens, which forms the impregnating agent with the water/residual water still contained in the bonded and/or reinforced fibres, the process according to the invention leads to the same product with significantly reduced effort as the known process of bonding individual layers of nonwoven fabric by hydroentanglement with subsequent complete drying and impregnation of the resulting cloth product.

A further advantage of the process according to the invention is that there are significantly fewer waste products to be disposed of or treated than in the known production of impregnated cloth products using hydroentanglement with subsequent removal of all the water from the bonded fibres/nonwovens, in which the process water used for reinforcing and recovered during subsequent drying must be completely reprocessed. In contrast, the process according to the invention produces significantly less or even no process water as a waste product, as this remains on the reinforced fibres and nonwovens as part of the impregnating agent, respectively.

Moreover, it is further advantageous that the process according to the invention allows immense energy savings compared to the production of cloth products using hydroentanglement known from the prior art, since the drying step for removing the water used for bonding the nonwovens is completely eliminated or at least shortened. In summary, the process according to the invention therefore offers energetic advantages, advantages in terms of time as well as economic advantages and conserves resources.

In general, the concentrate of the impregnating agent, which is applied to the fibres and/or nonwovens that are still impregnated with water and bonded and/or reinforced in the process according to the invention, can have any composition and can be adapted in its composition for the respective application. For example, the concentrate of the impregnating agent can be water-or oil-based and can also contain one or more further ingredients selected from the group comprising or consisting of alcohols, surfactants, antioxidants, preservatives, thickeners and ingredients with skin-caring, disinfectant and/or antimicrobial properties. Particularly preferably, the concentrate of the impregnating agent contains at least one surfactant.

According to one embodiment, the process according to the invention comprises the following steps:
a) Providing loose fibres or providing two or more nonwovens;
b) contacting the loose fibres or the two or more nonwovens with jets of water, the loose fibres bonding to form a fibre assembly or the two or more nonwovens bonding to one another;
c) contacting the fibres that are bonded to form a fibre assembly and impregnated with water from the hydroentanglement of step b), or the two or more nonwovens that are bonded to one another and impregnated with water from the hydroentanglement of step b) with a concentrate of an impregnating agent.

‘Impregnated’ in this context means that water from the hydroentanglement process adheres to the reinforced fibres or interconnected nonwovens in any desired manner.

Optionally, the process according to the invention has a step of partially drying the fibres that are bonded to a fibre assembly and impregnated with water from the hydroentanglement from step b) or the two or more interconnected nonwovens impregnated with water from the hydroentanglement from step b), wherein the drying step takes place after the hydroentanglement (step b) and prior to the application of the concentrate of an impregnating agent to the interconnected and/or reinforced fibres and/or nonwovens (step c) which are still impregnated with water.

It is preferred that the step of partially drying the fibres bonded together to form a fibre assembly and impregnated with water from the hydroentanglement of step b) or the two or more nonwovens bonded together and impregnated with water from the hydroentanglement of step b) includes measuring the moisture content of the fibres bonded together to form a fibre assembly or of the two or more nonwovens bonded together using a moisture meter.

Advantageously, the step of partially drying the fibres connected to form a fibre assembly and impregnated with water from the hydroentanglement from step b) or the two or more nonwovens bonded to one another and impregnated with water from the hydroentanglement from step b) is aborted when the moisture content detected by the moisture meter reaches or falls below a predetermined limit value for the residual moisture contained in the fibres connected to form a fibre assembly or in the two or more nonwovens connected to one another.

According to a preferred embodiment, the bonding of two or more nonwovens in the process according to the invention is carried out in a device comprising at least two drums in which the nonwovens to be bonded are treated with the water jets, the water jets in the first drum preferably impinging on the nonwovens to be bonded at a lower pressure than in the second drum.

Preferably, the water jets emerge from several nozzle strips in the first drum, wherein each nozzle strip in the first drum comprises several nozzles, wherein the water jets emerge from each nozzle belonging to the same nozzle strip at the same pressure and wherein the pressures of the water jets which emerge from nozzles belonging to different nozzle strips can be the same or different and are each between 10 and 80 bar.

Preferably, there are three nozzle strips in the first drum, wherein the pressure at which the water jets emerge from the nozzles of the first nozzle strip is preferably between 10 and 30 bar, wherein the pressure at which the water jets emerge from the nozzles of the second nozzle strip is preferably between 30 and 60 bar, and wherein the pressure at which the water jets emerge from the nozzles of the third nozzle strip is preferably between 60 and 80 bar.

It is further preferred that the water jets emerge from several nozzle strips in the second drum, wherein each nozzle strip in the second drum comprises several nozzles, wherein the water jets that emerge from each nozzle belonging to the same nozzle strip are at the same pressure and wherein the pressures of the water jets that emerge from nozzles belonging to different nozzle strips can be the same or different and are each between 30 and 100 bar, preferably between 60 and 100 bar, more preferably between 90 and 100 bar.

Optionally, the second drum has a dimpled structure, in particular a 2D dimpled structure, in order to achieve embossing of the resulting cloth product.

Further optionally, the second drum also has a moisture meter which is arranged to measure the moisture content adhering to the reinforced fibres or nonwovens after hydroentanglement. The optional drying in the process according to the invention is therefore preferably carried out in the second drum, with the moisture content in the reinforced fibres or nonwovens being measured during drying. The drying step is preferably stopped if the moisture meter determines that a predetermined proportion of the water adhering to the reinforced fibres or nonwovens after hydroentanglement has been removed, for example a proportion of up to 10%, up to 20%, up to 30%, up to 40% or up to 50%. After this optional step of partial drying, the concentrate of the impregnating agent is applied to the reinforced fibres or nonwovens and mixes with the water with which they are still impregnated after hydroentanglement or which still adheres to them after hydroentanglement to form the impregnating agent with which the impregnated cloth products produced by the process according to the invention are impregnated.

Finally, after reinforcing the fibres or bonding the nonwovens by contacting them with water jets and then applying the concentrate of impregnating agent, thereby obtaining the impregnated cloth products, the process according to the invention optionally still comprises final steps of cutting the obtained cloth products and folding the cut cloth products, which, after an optional packaging of the cut and folded cloth products, enables cloth-to-cloth removal from the packaging by the consumer.

The features of the invention disclosed in the foregoing description and in the claims may be essential, both individually and in any combination, for the realisation of the invention in its various embodiments.

1. Process for the production of cloth products impregnated with an impregnating agent, the process comprising reinforcing and/or bonding fibres and/or nonwovens by means of hydroentanglement, characterized in that, after hydroentanglement, a concentrate of an impregnating agent is applied to the fibres and/or nonwovens that are interconnected and/or reinforced and that are still impregnated with water, wherein the concentrate, together with water with which the interconnected and/or reinforced fibres and/or nonwovens are impregnated after hydroentanglement, forms the impregnating agent with which the impregnated cloth products are impregnated.

2. Process according to claim 1, wherein the concentrate of the impregnating agent is surfactant-containing, water-or oil-based and furthermore contains one or more further ingredients selected from the group comprising or consisting of alcohols, antioxidants, preservatives, thickeners and ingredients having skin-caring, disinfecting and/or antimicrobial properties.

3. Process according to claim 1, wherein it comprises the following steps:
(a) providing loose fibres or providing two or more nonwovens;
b) contacting the loose fibres or the two or more nonwovens with jets of water, whereby the loose fibres combine to form a fibre assembly or whereby the two or more nonwovens combine with one another;
c) contacting the fibres that are interconnected in a fibre assembly and impregnated with water from the hydroentanglement of step b), or the two or more nonwovens that are bonded to one another and impregnated with water from the hydroentanglement of step b) with a concentrate of an impregnating agent.

4. Process according to claim 1, characterized by a step of partially drying the fibres that are bonded to one another to form a fibre assembly and are impregnated with water from the hydroentanglement from step b) or the two or more nonwovens that are interconnected and impregnated with water from the hydroentanglement from step b), wherein the drying step takes place after the hydroentanglement and prior to the application of the concentrate of an impregnating agent to the fibres and/or nonwovens which are still impregnated with water and bonded together and/or reinforced.

5. Process according to claim 4, wherein the step of partially drying the fibres that are bonded to one another to form a fibre assembly and are impregnated with water from the hydroentanglement from step b) or the two or more nonwovens that are interconnected and impregnated with water from the hydroentanglement from step b) includes a measurement of the moisture content of the fibres that are bonded to one another in a fibre assembly or of the two or more nonwovens that are interconnected, the measurement using a moisture meter.

6. Process according to claim 5, wherein the step of partially drying the fibres that are bonded to one another to form a fibre assembly and are impregnated with water from the hydroentanglement from step b) or the two or more nonwovens that are interconnected and impregnated with water from the hydroentanglement from step b) is stopped when the moisture content detected by the moisture meter reaches a predetermined limit value for the residual moisture contained in the fibres bonded together in a fibre assembly or in the two or more nonwovens that are bonded together.

7. Process according to claim 1, wherein the water jets emerge from one or more nozzles before they hit the fibres or the two or more nonwovens.

8. Process according to claim 1, wherein the bonding of the fibres or of the nonwovens takes place in a device which has at least two drums in which the fibres or nonwovens to be bonded are treated with the water jets, the water jets in the first drum impinging on the fibres or nonwovens to be bonded at a lower pressure than in the second drum.

9. Process according to claim 8, wherein the water jets in the first drum emerge from a plurality of nozzle strips, wherein each nozzle strip in the first drum has a plurality of nozzles, wherein the water jets that emerge from each nozzle belonging to the same nozzle strip are at the same pressure and wherein the pressures of the water jets emerging from nozzles belonging to different nozzle strips can be the same or different and are each between 10 and 80 bar.

10. Process according to claim 8, wherein the water jets emerge in the second drum from a plurality of nozzle strips, wherein each nozzle strip in the second drum has a plurality of nozzles, wherein the water jets emerge from each nozzle belonging to the same nozzle strip at the same pressure and wherein the pressures of the water jets emerging from nozzles belonging to different nozzle strips can be the same or different and are each between 30 and 100 bar.

11. Process according to claim 8, wherein the second drum has surface-enlarging elements on its inner surface, whereby an embossing of the fibres or nonwovens bonded together takes place.

12. Process according to claim 8, wherein the moisture content of the fibres or nonwovens bonded together by the hydroentanglement is determined in the second drum.

13. Process according to claim 8, wherein the step of partially drying the fibres that are bonded in a fibre assembly and impregnated with water from the hydroentanglement from step b), or the two or more nonwovens that are interconnected and impregnated with water from the hydroentanglement from step b), takes place in the second drum, the moisture content in the fibres or nonwovens being measured during the drying.

14. Process according to claim 13, wherein the step of partial drying is stopped when it is determined by a moisture meter that a predetermined proportion of the water adhering to the reinforced fibres or nonwovens after hydroentanglement has been removed.

15. Process according to claim 14, wherein the step of partial drying is stopped when it is determined that a proportion of up to 10%, up to 20%, up to 30%, up to 40% or up to 50% of the water adhering to the reinforced fibres or nonwovens after hydroentanglement has been removed.
